(19) United States
(12) Patent Application Publication
(10) Pub. No.: US 2023/0182130 A1
(43) Pub. Date: Jun. 15, 2023

(54) MICROFLUIDIC SYSTEM FOR OIL SAMPLE ANALYSIS

(71) Applicants: Petróleo Brasileiro S.A. - Petrobras, Rio de Janeiro (BR); CNPEM - Centro Nacional De Pesquisa Em Energia E Materiais, Campinas (BR)

(72) Inventors: Rogerio Mesquita De Carvalho, Rio de Janeiro (BR); Gabriela Furlan Giordano, Campinas (BR); Aline Machado De Azevedo Novaes, Rio de Janeiro (BR); Igor Domingos Rodrigues, Rio de Janeiro (BR); Rafaella Magliano Balbi De Faria, Rio de Janeiro (BR); Angelo Luiz Gobbi, Campinas (BR); Renato Sousa Lima, Campinas (BR); Alexandre Ataíde Da Silva, Campinas (BR); Giulia Silva Da Silva, Campinas (BR); Rui Cesar Murer, Campinas (BR); Flavio Makoto Shimizu, Campinas (BR)

(21) Appl. No.: 18/077,814

(22) Filed: Dec. 8, 2022

(30) Foreign Application Priority Data
Dec. 9, 2021 (BR) 10 2021 024963 3

(51) Int. Cl. B01L 3/00; B01L 3/02

(52) U.S. Cl. CPC B01L 3/502707; B01L 3/022; B01L 3/0237; B01L 3/50273; B01L 2200/10; B01L 2200/18; B01L 2300/12; B01L 2300/023; B01L 2300/0663; B01L 2300/0816; B01L 2300/0861; B01L 2400/0406

(57) ABSTRACT

The present invention refers to a system comprising microfluidic devices of extraction, capacitance analysis, together with the smartphone-controlled potentiostat, consisting of a portable system that can be applied on offshore platforms and offering an analytical procedure that requires low levels of samples and chemical ingredients. In addition, the assembly provides the in loco, fast analysis of species having fouling features with high analytical frequency. The ability to carry out these analyzes in low BSW oils represents a strong analytical improvement view of the technical difficulties observed in traditional methods that use liquid-liquid extraction. Thus, the possibility of quickly predicting the ionic composition profile of these water samples becomes a strategy for monitoring, control and decision-making actions in the production chain, making it possible to establish more appropriate fouling inhibition strategies, enabling more proactive actions rather than reactive ones to be taken by the operator. Therefore, unscheduled production stops caused by fouling in the production system are prevented.

### FIELD OF THE INVENTION

The present invention deals with a microfluidic system that can be portable, which is applied to fluids produced on offshore or onshore platforms to extract aqueous phase-associated species in oil samples having low BSW values, making it possible to quickly predict the ionic composition profile of water samples for monitoring, control and decision-making actions in the production chain.

### DESCRIPTION OF THE STATE OF THE ART

Mineral deposition or fouling on surfaces is caused by the accumulation of inorganic salts. When present in oil and gas exploration and processing pipelines and equipment, fouling causes major operational issues, damage to pipes and safety risks to the environment and operators. This phenomenon is present not only in offshore wells but also in refineries and treatment units. Origin of salinity is due to the chemical composition of the so-called formation and injection waters.

Chemical analyzes of these fluids are carried out using well established techniques such as: atomic absorption, Inductively Coupled Plasma Optical Emission spectroscopy, ion chromatography and/or titrimetry However, it is not feasible to use these techniques on offshore platforms, since the analyzes are carried out in large and expensive equipment. Furthermore, before these analyzes are carried out, it is necessary to transfer ions from the oil dispersion to an aqueous matrix by liquid-liquid extraction (LLE). Reactors with pressure and temperature control are often used to obtain better outcomes in LLE. However, the method is time-consuming and has to be performed in properly equipped laboratories. Dilution with a known amount of water helps to separate the dispersed aqueous phase, but is of limited application in the case of matrices whose potential for forming stable emulsions is high.

Furthermore, separation of the aqueous phase at low BSW values is very difficult and cannot be performed by traditional techniques, such as centrifugation, without interfering with the sample. Even with liquid-liquid extraction, for example, a significant dilution factor exists, in addition to the difficulty in recovering the added water and a restricted extraction efficiency. As a result, analysis of the produced fluids involves sampling, unloading and analyzing the water on shore, which greatly increases the time and makes the “picture” of the system to reflect the past, making it difficult to adopt adequate strategies for the inhibition of fouling.

In carbon reservoirs, chemical reactions between the rock and the injected fluids are expected. These reactions can cause changes in the composition of the produced water. Dissolution of carbonates such as calcite and dolomite increases the concentration of calcium, magnesium and bicarbonate in the produced fluid. Then, composition of the produced water will not be just a mixture of the fluids present in the reservoir. Water composition has to be known while taking into account these rock and fluid interactions.

To adopt the best strategy for inhibiting fouling in wells and topside which, in the case of carbonates, depends on a more reliable modeling of the reactive transport, it is necessary to know the chemical composition of the fluids involved in the process: injection water, formation water and produced water.

Currently, the analysis to know the species having fouling features present in fluids produced on offshore platforms is carried out on shore, which takes a long time (for sampling, unloading the sample and analyzing) . This time has been long enough for the platform to stop production due to fouling in the systems before the chemical composition of the produced water is known so as the most adequate fouling inhibition strategy be defined. In addition to the time required to know the composition of the produced water, another drawback makes it difficult to obtain this information: the low BSW value makes it impossible to carry out chemical analysis, since it is not possible to separate a sufficient volume of water for the analysis. Recent analyzes have shown yet another issue: the change in the balance involved and alteration of the measured values. During water separation, a centrifugation procedure is employed.

BRESSAN, L. P. (2021) “Aplicações de dispositivos microfluídicos impressos em 3D por FDM em química”, Thesis (Doctorate) - University of Campinas, SP, presents bioanalytical applications for devices produced with a simple protocol to prepare 3D printed microfluidic channels for determining the levels of nitrite, total proteins and nitric oxide, in addition to visualizing microorganisms and the continuous flow synthesis of silver and gold nanoparticles. In this study, a protocol has also been developed to create support devices, designated as scaffolds, so that the pores produced are controlled directly through the printer software.

The article by CAMARGO, C.L. et al. (2017) “Use of smartphone for turbidimetric detection and control of turbulent microfluidic platform toward full automation of microemulsification-based method”, describes a method of turbidimetric detection and control of turbulent microfluidics for the purpose of fully automating the microemulsification (MEC)-based method. The method proved to be simple and autonomous, providing real-time results and remote data transmission capability. The device was made by polymerization and scaffold removal (PSR) to provide assisted heavy flow turbulence. This fully automated microfluidic platform was applied in the determination of ethanol in commercial alcoholic beverages, showing an improved accuracy and analytical frequency of MEC, being a potential alternative for point-of-use applications. This feature contributes to the use of this technology by non-specialists, providing in-situ measurements and real time readings.

The work by SOUSA, P. J. T. (2011) “Estudo e otimizaçgão de estruturas em PDMS para dispositivos microfluidicos”, Dissertation (Master in Micro/Nano technologies) - University of Minho, Portugal, describes an optimization study of the different stages of the entire process for the manufacture of PDMS structures and characterizes the produced structures. One of the optimization processes is the use of a thermoplastic material without the need for masks, alignment, exposure or development, requiring only a CAD design suitable for use by a printer. This study also discloses several applicability groups of PDMS in microfluidics.

In view of this, no state-of-the-art document discloses a portable microfluidic liquid-liquid extraction system used to separate the aqueous phase of oil samples with low BSW levels and that allows the classification and multidetermination of ions with fouling capacity present in the various fluids from carbon reservoirs, through multidimensional capacitive sensors, such as the one of the present invention.

Thus, the object of the present invention was to develop a system based on a microfluidic platform for the extraction of aqueous phase-associated species, considering oils with low water content. In addition, sensors based on the concept of electronic language and statistical treatment of data by machine learning (ML) are used for the classification and multidetermination of ions in water samples extracted from petroleum. This system is portable for use on offshore platforms.

Considering all of the system components, i.e. the microfluidic devices of extraction, capacitance analysis, together with the smartphone-controlled potentiostat, the assembly results in a portable system that can be applied on offshore platforms and offers an analytical procedure that requires low levels of samples and chemical ingredients. In addition, the assembly provides the in loco, fast analysis of species having fouling features with high analytical frequency.

The ability to carry out these analyzes in oils with low BSW levels represents a strong analytical improvement, in view of the technical difficulties observed in traditional methods that use liquid-liquid extraction. Assessment in low BSW oils is essential to characterize the formation water in the oil zone, since, at higher BSW values, there is already a greater presence of injected water and rock-fluid interaction. Therefore, the possibility of quickly predicting the ionic composition profile of these water samples becomes a strategy for monitoring, control and decision-making actions in the production chain.

From an economic point of view, it is possible to establish more adequate fouling inhibition strategies, enabling more proactive actions than reactive ones by the operator. Reactive transport models can be updated more frequently, making it possible to identify any anomalies or the presence of unmapped species. Therefore, unscheduled production stops caused by fouling in the production system are prevented.

### BRIEF DESCRIPTION OF THE INVENTION

The present invention deals with a microfluidic liquid-liquid extraction system for use in the separation of the aqueous phase of low BSW oil samples, which is currently carried out in on shore laboratories. Also, the invention enables the classification and multidetermination of ions having fouling capacity present in different fluids from carbon reservoirs through multidimensional capacitive sensors. Performance of the analysis can be facilitated by means of a portable smartphone-controlled potentiostat, not being limited to this, allowing the direct application in offshore environments and its use can be extended to other sectors that need quick monitoring of the saline composition of water samples, whether formation, production or injection water. The results will be used to monitor samples with a potential risk for fouling and will serve as the basis for a compilation of results to adjust the history of reactive transport modeling.

Such an analytical system can be applied in the analyses of fluids carried out both offshore and onshore.

### BRIEF DESCRIPTION OF THE DRAWINGS

The present invention will be described in more detail below, with reference to the attached figures which, in a schematic and non-limiting manner of the scope of the invention, represent examples of embodiments. In the drawings:

FIG. 1 illustrates soft lithography and 3D printing technologies, where (1) 2D view and (2) 3D view of the microfluidic chip associated with the extraction step;

FIG. 2 illustrates (A - 1) a sensor obtained by the PSR method, the stainless steel capillary probes are ready for use; (B - 2) and (C - 3) show the microfluidic sensors obtained with four and eight pairs of capacitors in parallel, respectively; and (D -4) shows a sensor obtained by soft lithography, the flat, interdigitated gold capacitors were deposited on glass plates by physical evaporation techniques in vapor phase, and their area and design were defined by photolithography. These represent the electronic language in different configurations;

- FIG. 3 illustrates the sequential scheme of operation of the extraction system associated with the electronic language.

### DETAILED DESCRIPTION OF THE INVENTION

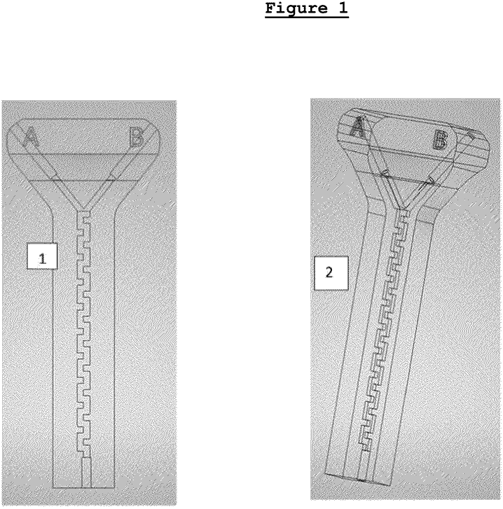

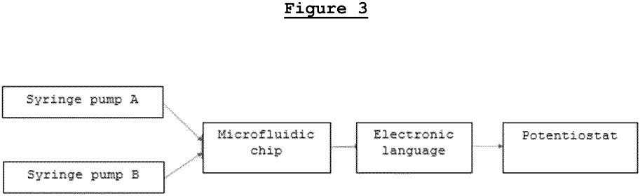

The portable system according to the present invention and illustrated in FIG. 3 comprises an electronic language, a potentiostat, microfluidic chips and two syringe pumps. Microfluidic chips of both liquid-liquid extraction and the electronic language system can be built by polymerization and scaffold removal (PSR), soft lithography and 3D printing technologies (FIG. 1 (1) 2D view and (2) 3D view).

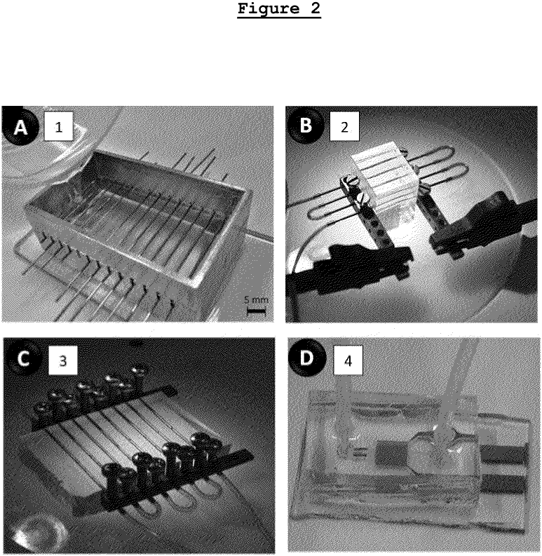

Among the materials used, silicone-based or thermoplastic polymers and epoxy or acrylic resins can be mentioned. In the sensor obtained by the PSR method (FIG. 2 (A)), the ready-to-use probes used were stainless steel capillaries. These probes were inserted on both sides of the microfluidic sensor channels in order to obtain gaps of about 200 µm between each other. These capillaries were short-circuited with copper pieces, obtaining an association of capacitors in parallel. Then, poly(vinyl chloride) hoses connected the capillaries to each other to complete the microfluidic circuit.

FIG. 2 (B) and (C) show the microfluidic sensors obtained with four and eight pairs of capacitors in parallel, respectively. In the sensor obtained by soft lithography, the flat, gold interdigitated capacitors (FIG. 2 (D)) were deposited on glass plates by physical evaporation techniques in vapor phase, and their area and design were defined by photolithography. Impedance analyzes were performed on portable potentiostats controlled by smartphones, so that analyzes can be performed on offshore platforms. Capacitances of the pairs of electrodes were calculated from the imaginary impedance considering the electrodes as ideally polarizable. Statistical treatment of data by ML was performed using the library algorithms Python.

### EXAMPLES

The following examples are presented to fully illustrate the nature of the present invention and how to practice the same, without, however, being considered limitative of its content.

### Example 1: Microfluidic Devices

The development of easy-to-manufacture microfluidic devices was obtained by using epoxy resins with the aid of a scaffold and also through stereolithography (SLA) 3D printing technology. These microfluidic devices were composed of intertwined channels having sizes of approximately 400 pm, which operated under a turbulent flow regime and presented characteristics of interest such as portability, low cost, capacity for ultra-fast extractions, low consumption of reagents and samples, chemical an d mechanical resistance and long shelf-life.

This device showed fruitful results concerning the extraction capacity of the aqueous fraction present in crude oil samples with BSW values lower than 1%. The extraction capacity was assessed through the analysis of water conductivity and Ca²⁺, Sr²⁺ (ICP-OES) and (SCU)²⁻ and Cl⁻ ions (ion chromatography). The experimental conditions of dilution with toluene, volume of diluent, flow rate, donor and receptor phase ratios were studied in order to maximize the cited extractive process.

### Example 2: Electronic Language

To quantify the ions in the water extracted by the device, an electronic language was developed, which consists of an electrochemical multidimensional sensor based on a single probe and universal capacitive detection in microfluidic devices. Fingerprints of the electronic language were obtained through a single capacitance experiment in low cost, ready-to-use probes (stainless steel capillaries), which form electric double layer capacitors (EDC) and were associated in parallel in the polydimethylsiloxane (PDMS) microfluidic device.

These devices presented low consumption of samples and were prototyped through a cleanroom-free green, scalable technique. The multidimensional data of differential capacitance of the electrical double layer (Cd) obtained by the sensor were confirmed through macroscopic measurements of capacitance and analysis by microscopy and spectroscopy methods. The sensor was used in the classification and multidetermination of mixtures of three Ba²⁺, Ca²⁺ and Cl⁻. Statistical treatment of the data was carried out using supervised ML techniques, which can be: partial least squares (PLS), random forests (RF), linear discriminant analysis (LDA), Sure Independence Screening and Sparsifying Operator (SISSO), among others. The application of SISSO enabled a simple and linear modeling of the data and selected only 2 Cd data (at different frequencies) that were associated with the ionic charge of the solution in the electric double-layer and the electrode material. Classification accuracy in validation samples was 100.0%. Regressions used for multidetermination showed a high correlation across the range of metal ion concentrations studied (R²> 0.9996), with an accuracy of 100.0%.

It should be noted that, although the present invention has been described in relation to the attached drawings, it may be subjected to modifications and adaptations by the skilled person, depending on the specific instance, but as long as it is within the inventive scope defined herein.

1. A PORTABLE MICROFLUIDIC SYSTEM FOR OIL SAMPLE ANALYSIS, characterized by comprising an electronic language, a potentiostat (which can also be controlled by a smartphone), microfluidic chips (extraction system) and syringe pumps, where probes are inserted into both sides of the microfluidic sensor channels.

2. The SYSTEM, according to claim 1, characterized in that the microfluidic chips for both liquid-liquid extraction and the electronic language are built by methods of polymerization and scaffold removal (PSR) soft lithography and 3D printing technologies.

3. The SYSTEM, according to claim 2, characterized in that the polymers are based on silicone or thermoplastics and epoxy or acrylic resins.

4. The SYSTEM, according to claim 2, characterized in that the sensor obtained by the PSR method, the probes are stainless steel capillaries.

5. The SYSTEM, according to claim 1, characterized in that the probes have a spacing of 200 µm between each other.

6. The SYSTEM, according to claim 4, characterized in that the capillaries are short-circuited with copper pieces, obtaining an association of capacitors in parallel.

7. The SYSTEM, according to claim 4, characterized in that poly(vinyl chloride) hoses connect the capillaries to each other to complete the microfluidic circuit.

8. The SYSTEM, according to claim 1, characterized in that the microfluidic sensors have four or eight pairs of capacitors in parallel.

9. The SYSTEM, according to claim 2, characterized in that the sensor obtained by soft lithography, the flat, gold interdigitated capacitors are deposited on glass plates by physical evaporation techniques in the vapor phase.
